(19) United States
(12) Patent Application Publication
(10) Pub. No.: US 2015/0157697 A1
(43) Pub. Date: Jun. 11, 2015

(54) LACTOFERRIN SUPPLEMENTATION AND DIARRHEA

(71) Applicant: Bing Wang, Wolli Creek (CN)

(72) Inventor: Bing Wang, Wolli Creek (CN)

(73) Assignee: NESTEC S.A., Vevey (CH)

(21) Appl. No.: 14/386,402

(22) PCT Filed: Mar. 20, 2012
(86) PCT No.: PCT/CN12/72594

§ 371 Date: Feb. 3, 2015

(51) Int. Cl. A61K 38/48; A23L 2/66; A23L 1/305

(52) U.S. Cl. CPC A61K 38/482; A23L 1/3056; A23L 2/66; A23V 2002/00

(57) ABSTRACT

The invention provides a composition comprising lactoferrin for use in the prevention, amelioration, or treatment of diarrhea, in particular non-infectious weaning diarrhea of infants or children.

The present invention relates in general to the field of intestinal health. In particular, the present inventors have found that a composition comprising lactoferrin can be used in the prevention, amelioration, or treatment of diarrhea. This finding is in particular important because long lasting diarrhea is seen as a common cause of mortality in infants and children.

Gut maturation during infancy relies heavily on nutrition support. Proper substantiation of the intestinal epithelia and gut microflora during early life has developmental programming significance as this may influence the responsiveness of the intestine to dietary, physiological or pathological challenges, such as sugar, fatty acids, and exogenous proteins later in life. Diarrhea is the manifestation of a disturbed gut environment, and could in turn negatively affect balanced gut ecology. Nutrient malabsorption and dehydration due to frequent diarrhea during the sensitive window of early infancy could impose long-term irreversible consequence for the infants. Diarrhea is one of the commonly seen symptoms in infants and children. If diarrhea lasts for more than two weeks it is seen as a common cause of mortality in infants and children.

Lactoferrin is one of the four major proteins in human milk and an iron-binding glycoprotein which has antioxidant activity and plays a role in the intestinal iron uptake and the regulation of immune response (Wong, et al., J Dairy Res, 1998. 65(4): p. 697-701; Dial, et al., Dig Dis Sci, 1998. 43(12): p. 2750-6; Lindmark-Mansson, et al., Br J Nutr, 2000. 84 Suppl 1: p. S103-10.). It is estimated that the lactoferrin concentration is about 9.7 g/L in the colostrum, and 2-3 g/L in mature milk (Ronayne de Ferrer, et al., J Am Coll Nutr, 2000. 19(3): p. 370-3). Lactoferrin derivatives have been shown to be an effective alternative to feeding antibiotics for enhancing growth performance in piglets weaned at age 21 days.

Recent studies have again shown the beneficial effects of mother milk feeding in preventing morbidity and mortality from diarrhea in both human infants and other mammals.

Weaning age babies frequently suffer from diarrhea, as maternal antibodies either from pregnancy or human milk for protection against diseases have significantly decreased at the age of four to six months. For example, if weaning foods are introduced to babies at a younger age than 4-6 months, this may result in infant diarrhea. Typical occasions are changes in milk feeding patterns from breast-milk to formula-milk, or from one brand infant formula to another brand. Diarrheal diseases are the 2nd commonest illnesses after respiratory infections that have the greatest negative impact upon the growth of infants and young children. Diarrhea and possibly resulting malnutrition is responsible for a significant proportion of the 13 million deaths among infants and children under 5 years of age worldwide each year. Thus the prevention of weaning diarrhea is of high importance for public health and infant nutrition to ensure a healthy development and no growth retardation for children.

Consequently, there is a need for natural ways to treat of prevent the occurrence of diarrhea, in particular in infants and children.

The present inventors have addressed this need. It was consequently the objective of the present invention to improve the state of the art, and specifically to provide the art with a natural, safe and side-effect free way to prevent or treat diarrhea and/or to reduce its duration and/or severity, in particular in infants and children.

The present inventors were surprised to find that the subject matter of the independent claims achieves the objective of the present invention. The subject matter of the dependant claims further develops the idea of the present invention.

In particular the inventors could demonstrate that the administration of lactoferrin during the weaning phase, infancy, and/or childhood allowed reducing the occurrence of diarrhea, its duration and severity.

The inventors have chosen new born piglets as an animal model because the pig's digestive system shares a similar physiology and anatomical structure with human infants and has comparable nutrient requirements (Wijtten et al., Br J Nutr. 2011 April; 105(7):967-81. Epub 2011 Feb. 8; 7. Moughan et al., World Review of Nutrition & Dietetics 1992; 67:40-113).

Both newborn piglets and low-birth-weight and newborn infants are vulnerable to developmental deficits. This makes the piglet ideally suited for the coordinated nutritional, metabolic and molecular investigations described in this application.

Without wishing to be bound by theory the inventors presently believe that the surprising beneficial effect of lactoferrin that was observed in the present invention is due to the ability of a lactoferrin molecule to bind two ferric iron molecules. This might help to prevent biofilm formation of pathogenic microbes. As a consequence, lactoferrin may be considered a part of the protection machinery conferred to the infants by mothers.

Consequently, the present invention relates in part to a composition comprising lactoferrin for use in the prevention, amelioration, or treatment of diarrhea.

The present invention also relates to the use of lactoferrin in the preparation of a composition to treat, prevent or ameliorate diarrhea.

For example, the diarrhea may be a non-infectious diarrhea. Non-infections diarrhea may be caused by food poisoning, food allergies, food additives, malabsorption syndromes, for example gluten or lactose intolerance, antibiotic administration, heredity or travel.

The diarrhea may also be an infectious disorder. Infectious diarrhea may be caused by parasites, bacterial or viral infections, for example.

The present inventors have found that the composition of the present invention is in particular useful to treat, prevent or ameliorate weaning diarrhea.

Weaning diarrhea occurs often—but not only—in areas of insufficient sanitation and represents a major cause of death of infants of 6-24 months in developing countries. Weaning from maternal milk results in exposing the infant to new organisms and possible food deterioration while at the same time passively transferred IgA is lost.

The present inventors have shown that the administration of the composition of the present invention leads to a reduction of the duration of diarrhea. This is very important as short durations of diarrhea are normally uncomfortable but not dangerous, chronic diarrhea can have serious consequences, may lead to malnutrition and may be life threatening in particular for infants. Reducing the duration of diarrhea is therefore an important objective.

A diarrhea that lasts for more than 2 weeks is considered persistent or chronic. A diarrhea is considered to be acute, when there is passage of three or more loose stools in a 24 hour period, and when the diarrhea has duration of less than 2 weeks.

The composition of the present invention is also effective in reducing the severity of diarrhea. A severe diarrhea is not only very uncomfortable but often leads to dehydration next to all its other negative effects. Hence reducing the severity of diarrhea is another important objective that the subject matter of the present invention achieves as the inventors could demonstrate. Consequently, the composition of the present invention may also be for use in reducing the severity of diarrhea.

Diarrhea is characterized by an increase in the frequency of bowel movements compared to normal. Among healthy individuals the maximum number of daily bowel movements may be approximately three, so, for example, diarrhea can be understood as any number of stools greater than three.

Frequent bowel movements are uncomfortable and may have negative consequences for health. Advantageously, the subject matter of the present invention allows it to reduce the frequency of bowel movements. Hence, the composition of the present invention may be for use in reducing the frequency of diarrhea.

Diarrhea may be triggered by many factors, both inflammatory and non-inflammatory. The inventors could show that lactoferrin administration allows delaying the onset of diarrhea. This is beneficial as it allows infants and young children to mature further before the onset of diarrhea. A more mature an organism can usually better cope with the consequence of diarrhea. Consequently, the composition of the present invention may be for use in delaying the onset of diarrhea.

The composition of the present invention may be any composition comprising lactoferrin. Typically it is a composition intended for human or animal consumption.

The composition may contain lactoferrin naturally.

For example, the composition may be enriched in lactoferrin. A composition is enriched in lactoferrin if it contains more lactoferrin that it would naturally.

A composition may be considered as enriched if it contains lactoferrin in an amount corresponding to at least 110 weight-%, 120 weight-%, 150 weight-%, 170 weight-%, 200 weight-%, or 500 weight-%, of its natural lactoferrin content.

This may be achieved by a processing the composition or a part thereof to increase the lactoferrin content. Lactoferrin can also be added to the composition. Consequently, the composition may be supplemented with lactoferrin.

Lactoferrin may be provided as pure compound, but may also be provided as a fraction of a natural food product, for example. As such lactoferrin may be provided as a milk fraction.

The composition of the present invention may be administered to humans or animals in need thereof.

For example, the composition may be to be administered to infants or children, for example infants delivered by caesarian section, premature birth infants or IUGR infants.

Infants are children under the age of 12 months.

Children may be for example from 1-12 years old, from 1-6 years old or from 1-3 years old.

Infants delivered by caesarian section, premature birth infants or IUGR infants are believed to be at an increased risk for developing diarrhea, as immature intestinal barriers, permeability, motility and the enteric nervous system significantly influence gut function. Consequently, the composition of the present invention may be in particular beneficial for these infants.

The composition of the present invention contains lactoferrin in an effective dose. This effective dose can be readily determined by those of skill in the art.

Generally it was found that the effect of lactoferrin in the prevention, treatment or amelioration of diarrhea follows a dose response curve.

In therapeutic applications, compositions are administered in an amount sufficient to at least partially cure or arrest the symptoms of the disease and its complications. An amount adequate to accomplish this is defined as “a therapeutically effective dose”. Amounts effective for this purpose will depend on a number of factors known to those of skill in the art such as the severity of the disease and the weight and general state of the patient.

In prophylactic applications, compositions according to the invention are administered to a patient susceptible to or otherwise at risk of a particular disease in an amount that is sufficient to at least partially reduce the risk of developing a disease. Such an amount is defined to be “a prophylactic effective dose”. Again, the precise amounts depend on a number of patient specific factors such as the patient's state of health and weight.

The composition of the present invention is administered in a therapeutically effective dose or in a prophylactic effective dose.

For example, the composition may comprise lactoferrin in an amount corresponding to about 5-500 mg lactoferrin/kg body weight/day, 15-400 mg lactoferrin/kg body weight/day, 50-350 mg lactoferrin/kg body weight/day, and/or 100-300 mg lactoferrin/kg body weight/day.

As the dose, the ideal frequency of administration can be readily determined by those of skill in the art.

For an optimal effect a regular administration might be preferred. For example, the composition of the present invention may be to be administered at least daily, for example at least 3 times daily or at least 5 times daily.

A regular lactoferrin administration will allow keeping the lactoferrin level more constant, resulting in a prolonged protective effect.

The composition may be to be administered to humans or animals. The animals may be companion animals or domestic animals, for example. Diarrhea in companion animals or domestic animals is not only a problem for the animal but also for person taking care of the animal and the composition of the present invention has beneficial effects here.

Generally, the composition may be any kind of composition as long as it is consumable for humans or animals.

For example, the composition may be selected from the group consisting of an infant feeding formula, a nutritional formula, a formula for feeding newborn animals, a nutritional supplement, a food additive, a foodstuff and a drink.

In one embodiment the composition of the present invention is an infant feeding formula. This infant feeding formula may be intended for infants that are at an increased risk for developing diarrhea.

Those skilled in the art will understand that they can freely combine all features of the present invention described herein, without departing from the scope of the invention as disclosed.

In particular, features described for the uses of the present invention may be applied to the composition of the present invention and vice versa.

Further advantages and features of the present invention are apparent from the following Examples and Figures.

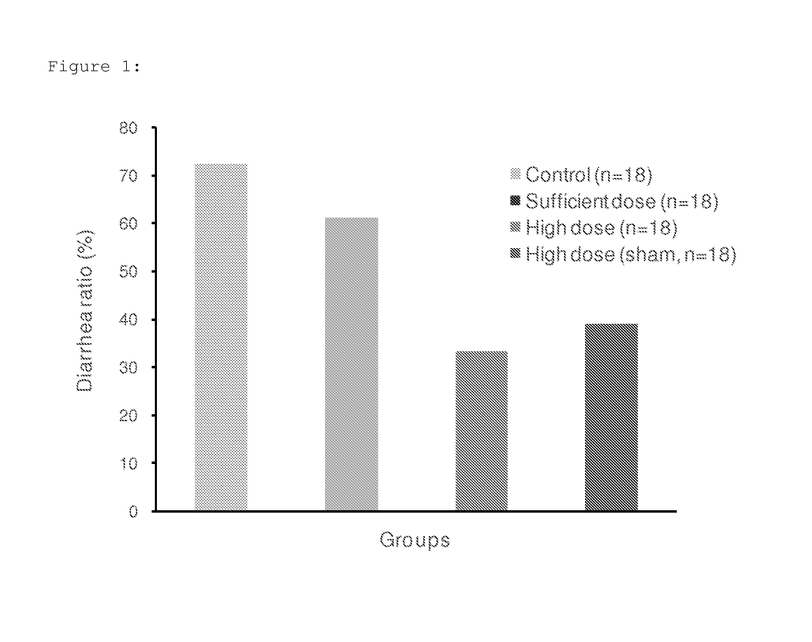

FIG. 1 shows the effect of lactoferrin supplementation on the non-infectious diarrhea ratio in formula fed piglets during the first 38 days after birth.

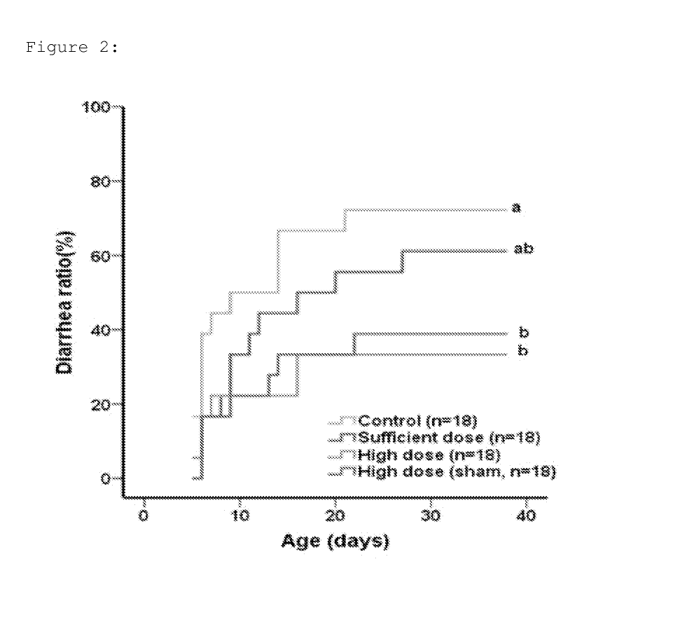

FIG. 2 shows the survival curve for the incidence of non-infectious diarrhea in piglets during the first 38 days after birth. Lines with different letters were significantly different (P<0.05, Kaplan-Meier survival curve) based on Log-rank pairwise test.

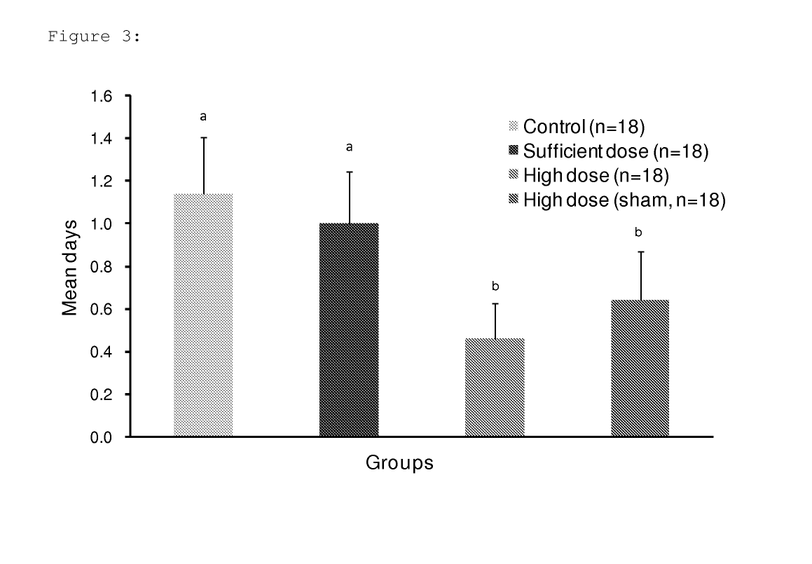

FIG. 3 shows the mean days of duration weaning (non-infectious) diarrhea in formula fed piglets during the first 38 days after birth. Values are means±SEM of 18 piglets. The columns with different letters were significantly different (P<0.05) based on Univariate ANOVA Bonferroni adjustment for multiple comparisons.

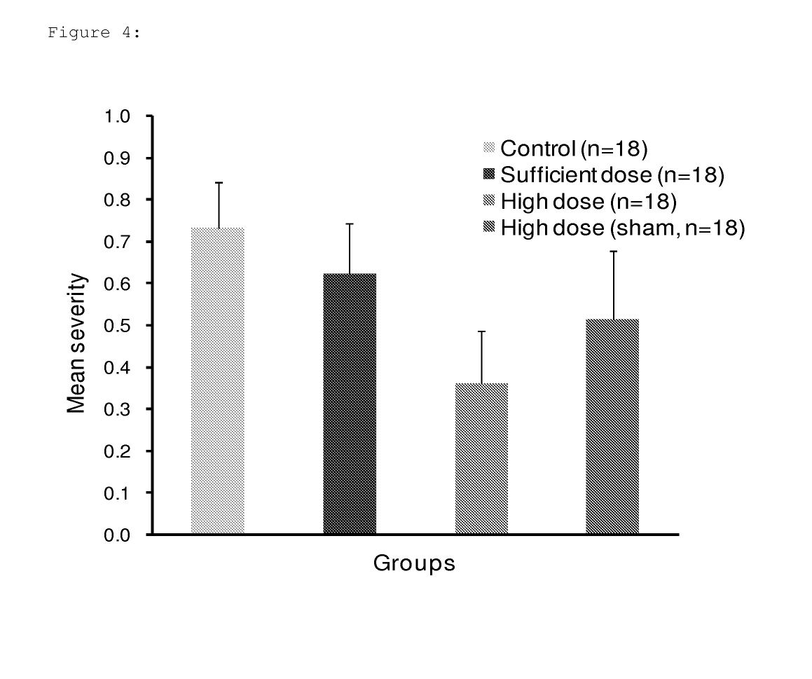

FIG. 4 shows the effect of lactoferrin supplementation on the severity of weaning (non-infectious) diarrhea in formula fed piglets during the first 38 days after birth (means±SEM).

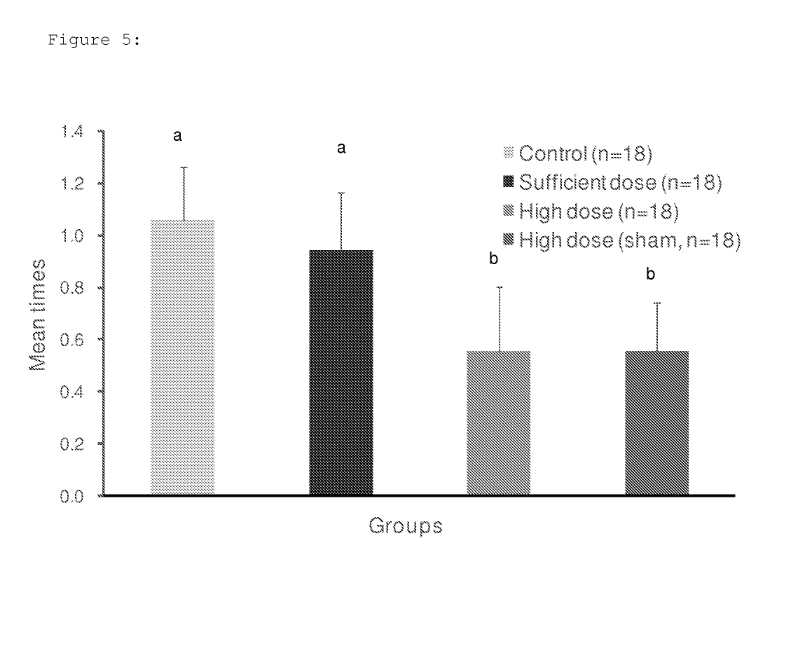

FIG. 5 shows that lactoferrin supplementation significantly reduces the mean incidence of non-infectious diarrhea in formula fed piglets with dose response during the first 38 days of life P=0.017, General linear model (Univariate ANOVA) with Bonferroni's adjustment for the use of antibiotic as a covariance). Values are means±SEM of 18 replicate piglets. The columns with different letters were significantly different (P<0.05) based on Bonferroni adjustment.

### EXAMPLES

### Experimental Procedures

### Animals

Seventy-two 3-day-old male domestic piglets (Sus scorfa Landrace×Large White F1) from 5 litters were purchased from a commercial piggery in Xiamen. The piglets were randomly allocated to 1 of 4 treatments according to weight and litter. All animals were housed in pairs in a temperature controlled environment with a 12-h light (08:00˜20:00) and dark (20:00˜08:00) cycle. The home pens contained a “nest” (a rubber tire covered with a clean towel), a heat lamp over the nest and an identical woody toy hang in the home pen. The maximum capacity of holding piglets at the behavior lab were 16, thus 5 trials (10˜16 piglets/trial) were conducted to reach 16˜18 piglets/group (Tab 1). The piglet behavior (including home pens) were monitored for 24-h with 7 camera surveillance systems. Two 3 day-old piglets were collected in each trial and euthanized as baseline control (n=10). The study protocol was approved by the Xiamen University Animal Ethics Committee.

TABLE 1
Grouping assignment

Group

| | Group 1 | Group 2 | Group 3 | Group 4 |
|---|---|---|---|---|
| Behavioral | Yes | Yes | Yes | No |
| test | | | | |
| Dosage | Control | Low | High | High |
| Total | 18 | 18 | 18 | 18 |
| piglet/group | | | | |

### Lactoferrin Supplementation

Bovine milk lactoferrin was purchased from DMV international. Piglets were fed a standard sow milk replacer containing protein of soy/whey/casein (50:38:12) from 3 days old to 38 days old. The amount of lactoferrin in the final milk varied according to the groups: 0.05 g/L (group 1, the control group with no added lactoferrin), 0.5 g/L (group 2, sufficient dose), 1 g/L (group 3, high dose). All piglets in groups 1-3 were exposed to learning challenges. Group 4 was supplemented with lactoferrin at the same dose as group 3, 1 g/L, but was not exposed to learning challenges (as sham group).

These concentrations represented an approximate intake of lactoferrin in the control, sufficient dose and high dose group were 15, 145 and 285 mg/kg body weight/day, respectively. The pig milk replacers were formulated so that total protein intake remained the same irrespective of the amount of added lactoferrin. To maintain normal growth rates, the piglets received 285 mL milk/kg body wt/day in the first 2 wk of the study, and 230 mL/kg body weight/day in the remaining weeks. Feeding times were at 08:00, 13:00, 18:00, and 22:30, with an extra 50 mL milk/pig supplied at the last feeding. Body weight was weighted every morning before feeding using a digital scale (PRIS-Scale model: XK 3116). The milk intake, health status, stool frequency, stool score, medication of piglets were recorded daily. Diarrhea was defined as having three or more stools than usual, with very loose or mucous.

### Evaluated Parameters

1. Incidence of diarrhea.

It is calculated as (number of piglets with diarrhea/total number of piglets in each group)×100%

### 2. Duration of Diarrhea

It is defined as total length (days) of diarrhea within a group

### 3. Severity of Diarrhea

Severity of diarrhea was quantified using a faecal consistency score based on a scale from 0 to 3 (0=normal faeces, 1=soft faeces, 2=soft faeces with white or yellow mucus, 3=watery diarrhea)

### 4. Mean Incidence of Diarrhea

It is defined as the total number of diarrheas occurred in each piglet within a group.

### Statistical Analysis

Differences in incidence of diarrhea (ratio number of piglet's diarrhea vs non-diarrhea) were compared using Chi-square Tests. The differences in the onset of diarrhea were compared using Kaplan-Meier survival analysis with Cox regression to examine potential covariates that may influence diarrhea. Comparisons between means of the average duration, severity, and average incidence of diarrhea in different groups were performed using the general linear model (Univariate ANOVA) with Bonferroni adjustment for multiple comparisons where appropriate. All statistical analyses were completed using SPSS for Windows 11 and 12 Inc, Chicago. A significance level of 0.05 was used.

### Results

1. Lactoferrin supplementation reduces the incidence of diarrhea in formula fed piglets during the first 38 days after birth FIG. 1 shows the effect of lactoferrin supplementation on the non-infectious diarrhea ratio in formula fed piglets during the first 38 days after birth. The diarrhea ratio was calculated as the percentage of piglets that had symptoms of diarrhea during the first 38 days after birth. The diarrhea ratio has a trend to decrease with the increase of lactoferrin supplementation concentration.

2. Lactoferrin supplementation significantly delayed the onset of diarrhea in formula fed piglets during the first 38 days of life using Kaplan-Meier survival curve analysis (P<0.05, FIG. 2). Compared with control group, the onset of non-infectious diarrhea was delayed in formula fed piglets in response to lactoferrin in a dose-dependent manner (P=0.044, Log-rank test).

3. Lactoferrin supplementation reduces the duration of weaning (non-infectious) diarrhea in formula fed piglets during the first 38 days after birth. The duration of diarrhea was calculated as the average days of diarrhea symptoms occurred in each group. Compared with control group (1.14±0.27 days), the duration of weaning (non-infectious) diarrhea decreased to 0.46±0.17 days with learning challenge and 0.64±0.23 day without learning challenge group when we administrated lactoferrin at dose level of 1 mg/L.

Also there was significant dose responses effect in reducing the duration of weaning (non-infectious) diarrhea after lactoferrin supplementation using General linear model (Univariate ANOVA) with Bonferroni's adjustment for multiple comparisons, antibiotic was used as the covariance (P=0.03, FIG. 3).

4. Lactoferrin supplementation reduces the severity of weaning (non-infectious) diarrhea in formula fed piglets during the first 38 days after birth (FIG. 4). Severity of diarrhea was quantified using a faecal consistency score based on a scale from 0 to 3 (0=normal faeces, 1=soft faeces, 2=soft faeces with white or yellow mucus, 3=watery diarrhea and color 1=yellow, 2=yellow or white mucous stool, 3=watery stool).

5. Lactoferrin supplementation reduces the mean incidence of weaning (non-infectious diarrhea) in formula fed piglets during the first 38 days after birth (FIG. 5). Compared with control group (1.06±0.21 day), supplemental lactoferrin reduces the incidence of weaning (non-infectious) diarrhea in formula fed piglets (0.56±0.25 day in 1.0 mg/L with learning group, and 0.56±0.18 day in 1.0 mg/L without learning group) during the first 38 days after birth, and the dose response was significant (P=0.017) based on the General linear model (Univariate ANOVA) with Bonferroni's adjustment for multiple comparisons, the use of antibiotic was taken as a covariance.

### CONCLUSION

Dietary supplementation of lactoferrin not only decreased the incidence and severity of diarrhea, but also delayed the onset of diarrhea for newborn piglets. These findings show that dietary lactoferrin supplementation improves gut comfort and is beneficial for the gut maturation process for young mammals.

1. A method for prevention, amelioration, or treatment of diarrhea comprising the step of administering a composition comprising lactoferrin to a subject in need of same.

2. Method in accordance with claim 1, wherein the diarrhea is a non-infectious diarrhea.

3. Method in accordance with claim 1, wherein the diarrhea is a weaning diarrhea.

4. Method in accordance with claim 1, for use in reducing the duration of diarrhea.

5. Method in accordance with claim 1, for use in reducing the severity of diarrhea.

6. Method in accordance with claim 1, for use in reducing the frequency of diarrhea.

7. Method in accordance with claim 1, for use in delaying the onset of diarrhea.

8. Method in accordance with claim 1, wherein the composition is supplemented with lactoferrin.

9. Method in accordance with claim 1, wherein the lactoferrin is provided as a milk fraction.

10. Method in accordance with claim 1, wherein the subject is an infant or child.

11. Method in accordance with claim 1, wherein the composition comprises lactoferrin in an amount corresponding to about 5-500 mg Lactoferrin/kg body weight/day.

12. Method in accordance with claim 1, wherein the composition is administered at least daily.

13. Method in accordance with claim 1, wherein the subject is a human or animal.

14. Method in accordance with claim 1, wherein the composition is selected from the group consisting of an infant feeding formula, a nutritional formula, a formula for feeding newborn animals, a nutritional supplement, a food additive, a foodstuff and a drink.
